(19) United States
(12) Patent Application Publication
(10) Pub. No.: US 2007/0129329 A1
(43) Pub. Date: Jun. 7, 2007

(54) Stabilized pharmaceutical composition of pramipexole and method of preparation thereof

(75) Inventors: Rajesh Kshirsagar, Vadodara (IN); Krishnakant Gandhi, Vadodara (IN); Amol Burkul, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: ALEMBIC LIMITED, Vadodara (IN)

(21) Appl. No.: 11/607,760

(22) Filed: Dec. 1, 2006

(30) Foreign Application Priority Data
Dec. 2, 2005 (IN) 1492/MUM/2005

(51) Int. Cl. A61K 31/724

(52) U.S. Cl. 514/58

(57) ABSTRACT

Stabilized pharmaceutical compositions comprising pramipexole or pharmaceutically acceptable salts thereof and one or more dextrins and to methods of preparation of the same. The said stabilized composition is in form of tablets comprising pramipexole dihydrochloride, β-cyclodextrin and one or more pharmaceutically acceptable excipients. A process for preparing the stabilized tablet composition, the process comprising dissolving pramipexole dihydrochloride along with polyvinyl pyrrolidone in suitable solvent; granulating blend of cyclodextrin and other excipients with above solution as granulating fluid; drying of above formed granules; lubricating granules with glidants and anti-adherents; compressing granules using suitable tablet equipment. A further process of preparing a stabilized tablet composition the process comprising preparing pramipexole dihydrochloride-β-cyclodextrin inclusion complex; admixing prepared inclusion complex with other excipients; granulating using either dry granulation process or wet granulation process or direct compression; drying, sifting and lubricating, formed granules; compressing granules using suitable tablet equipment to form tablet. A method of packaging the stabilized pharmaceutical composition comprising including oxygen absorbers or inert gas in the packaging system comprising the composition

### FIELD OF INVENTION

The present invention relates to stabilized pharmaceutical compositions comprising pramipexole or pharmaceutically acceptable salts thereof and one or more dextrins and to methods of preparation of the same.

### BACKGROUND OF INVENTION

Pramipexole, disclosed in U.S. Pat. No. 4,886,812 is a dopamine D2 receptor agonist useful in treatment of Parkinson's disease. The most commonly used salt of pramipexole is pramipexole dihydrochloride which is (S)-2-amino-4,5,6,7-tetrahydro-6-(propylamino) benzothiazole dihydrochloride monohydrate (FIG. 1). Its empirical formula is C₁₀H₁₇N₃S.2 HCl.H₂O and molecular weight is 302.27. Pramipexole dihydrochloride is a white to off-white powder substance. Pramipexole as its dihydrochloride salt is commercially available as MIRAPEX tablets of Pharmacia & Upjohn. These are immediate-release tablets in 0.125 mg, 0.25 mg, 0.5 mg, 1.0 mg and 1.5 mg strengths, designed for oral administration of a single tablet three times per day to provide a daily dose of 0.375 to 4.5 mg. Doses herein are expressed in amounts of pramipexole dihydrochloride monohydrate unless otherwise specified; 1.0 mg pramipexole dihydrochloride monohydrate is equivalent to about 0.7 mg pramipexole base.

[image]

The NDA submitted to United States FDA for MIRAPEX tablets discloses, that in solid state, pramipexole dihydrochloride itself has good stability but the tablet formulation is susceptible to photo degradation. Pramipexole dihydrochloride in the presence of excipients degrades resulting in a fall in the potency of the composition on storage at different stability conditions. This ultimately affects the shelf life of pramipexole compositions.

Therefore, there is a need in the art for stabilized pharmaceutical compositions of pramipexole.

### OBJECTS OF THE INVENTION

An object of the invention is to provide a stabilized pharmaceutical composition comprising pramipexole or pharmaceutically acceptable salts thereof and one or more dextrins.

Another object of the invention is to provide methods of preparation of stabilized pharmaceutical compositions comprising pramipexole or pharmaceutically acceptable salts thereof and one or more dextrins

Another object of the invention is to provide methods of packaging stabilized pharmaceutical compositions comprising pramipexole or pharmaceutically acceptable salts thereof and one or more dextrins

### SUMMARY OF THE INVENTION

According to one aspect of the present invention there are provided stabilized pharmaceutical compositions comprising pramipexole or pharmaceutically acceptable salts thereof and one or more dextrins.

According to another aspect of the invention there is provided a stabilized pharmaceutical composition comprising pramipexole or a pharmaceutically acceptable salt thereof and one or more dextrins and further comprising one or more pharmaceutically acceptable excipients.

According to a further aspect there are provided methods of preparing stabilized pharmaceutical compositions comprising pramipexole or pharmaceutically acceptable salts thereof and one or more dextrins.

According to yet another aspect there are provided methods of packaging stabilized pharmaceutical compositions comprising pramipexole or pharmaceutically acceptable salts thereof and one or more dextrins in a suitable packaging system with means to reduce to reduce oxygen content within the packaging system comprising the compositions.

In another aspect there is provided a method of treating disorders in a mammal in need thereof wherein pramipexole or pharmaceutically acceptable salts thereof are effective by administering to the mammal a stabilized pharmaceutical composition comprising pramipexole or a pharmaceutically acceptable salt thereof and one or more dextrins. Examples of such disorders include but are not limited to Parkinson's disease, restless legs syndrome and the like.

### DETAILED DESCRIPTION OF THE INVENTION

The present inventors have surprisingly discovered that pramipexole or its pharmaceutically acceptable salt can be stabilized in a pharmaceutical composition through the incorporation of one or more dextrins in the composition.

In a preferred embodiment, pramipexole is used in the form of its dihydrochloride salt also known as pramipexole dihydrochloride monohydrate.

The percentage of one or more dextrins in the stabilized composition can vary between 0.01% to 95% w/w of the composition.

The term “stabilized” as used herein, refers to the percentage potency of pramipexole retained in the composition after exposure to accelerated stability conditions, for example 40° C. with 75% relative humidity and/or 50° C. and/or the like for periods between 0-24 weeks. The percentage potency of pramipexole retained is 90%, preferably 95%. Alternatively, the term “stabilized” also refers to the percentage of degradation of pramipexole observed in the composition after exposure to accelerated stability conditions, for example 40° C. with 75% relative humidity and/or 50° C. and/or the like for periods between 0-24 weeks. The percentage degradation of pramipexole is not more than 10%, preferably not more than 5%.

The term “dextrin”, as used herein refers to any polymer which is produced by the hydrolysis of starch. Examples of dextrins includes but are not limited to α-cyclodextrin, β cyclodextrin, γ-cyclodextrin, alkyl or hydroxyalkyl derivatives thereof, such as (2,6-di-o-methyl)-β-cyclodextrin, randomly methylated β-cyclodextrin and hydroxypropyl β-cyclodextrin, sulpho-butyl-ether β-cyclodextrin, maltodextrin, amylodextrin and the like.

In preferred embodiment the dextrin is cyclodextrin. In still preferred embodiments the cyclodextrin is β-cyclodextrin. Thus in a preferred aspect there is provided a stabilized pharmaceutical compositions comprising pramipexole or pharmaceutically acceptable salts thereof and β-cyclodextrin.

The cyclodextrin may further be present in the stabilized composition as co-excipient, or as an inclusion complex with pramipexole. In preferred embodiments, where the cyclodextrin is a co-excipient, the level of cyclodextrin in stabilized composition is between 1% to 60% w/w of the composition and still more preferably between 20% to 40% w/w of the composition. In case of the inclusion complex the molar ratio of pramipexole to cyclodextrin can vary but is preferably between 1:0.25 to 1:4 and still more preferably 1:1. The level of inclusion complex in the composition can vary between 0.01% to 95% w/w of the composition.

The composition can be formulated for oral, nasal, ocular, urethral, buccal, transmucosal, intramuscular, intravenous, vaginal, topical, rectal delivery or the like. In a preferred embodiment, the composition is meant for oral delivery. In a still preferred embodiment, the composition is a tablet.

The composition can be formulated for delivering pramipexole or pharmaceutically acceptable salts thereof in a variety of release profiles. For example pramipexole or pharmaceutically acceptable salts thereof may be released immediately or its release may be modified e.g. controlled release, pulsatile release, extended release, delayed release, targeted release, targeted delayed release, or combinations thereof.

The stabilized pharmaceutical compositions comprising pramipexole or its pharmaceutically acceptable salt and one or more dextrin can further comprise one or more pharmaceutically acceptable excipients.

The phrase “pharmaceutically acceptable excipients,” as used herein, includes all physiologically inert additives used in pharmaceutical dosage forms.

In preferred embodiments the pharmaceutically acceptable excipients are those inert additives, which are required in the tablet dosage form. Examples of such excipients include but are not limited to binders, diluents, lubricants/glidants, coloring agents, and the like, or mixtures thereof.

Examples of binders include but are not limited to methyl cellulose, hydroxypropylcellulose, polyvinylpyrrolidone, gelatin, gum arabic, ethylcellulose, polyvinyl alcohol, pullulan, pregelatinized starch, agar, tragacanth, sodium alginate, propylene glycol, and the like, or mixtures thereof. In preferred embodiments the binder is polyvinylpyrrolidone.

Examples of diluents include but are not limited to calcium carbonate, calcium phosphate dibasic, calcium phosphate tribasic, calcium sulfate, microcrystalline cellulose, silicified microcrystalline cellulose, powdered cellulose, kaolin, lactitol, lactose, mannitol, sorbitol, starch, starch pregelatinized, sucrose, sugar compressible, sugar confectioners, and the like, or mixtures thereof. In preferred embodiments, the diluent is a mixture of starch, mannitol and microcrystalline cellulose.

Examples of lubricants and glidants include but are not limited to silicon dioxide, colloidal silicon dioxide, stearic acid, magnesium stearate, calcium stearate, talc, hydrogenated castor oil, sucrose esters of fatty acids, microcrystalline wax, yellow beeswax, white beeswax and the like, or mixtures thereof. In preferred embodiments colloidal silicon dioxide and/or magnesium stearate can be used as the lubricant and glidant.

The coloring agents may be selected from any FDA approved color agents for oral use. Examples of coloring agents include but are not limited to alumina, calcium carbonate, talc, titanium dioxide, aluminum powder, zinc oxide, FD&C Blue No. 1, FD&C Blue No. 2, D&C Blue No. 4, FD&C Green No. 3, D&C Green No. 5, D&C Red No. 21, D&C Red No. 22, D&C Red No. 27, D&C Red No. 28, D&C Red No. 30, FD&C Red No. 40, FD&C Yellow No. 5, FD&C Yellow No. 6 and the like.

The above listed excipients should be taken as merely exemplary and not limiting of the types of excipients that can be included in the compositions of the present invention. Also it has to be appreciated that there is considerable overlap between the above listed excipients in common usage since a given excipient is commonly used for any of several apparent functions. The amount of each excipient employed may vary within the ranges well known to those skilled in the art.

The stabilized composition of present invention can be prepared by a suitable method well known in the art. For example tablets can be prepared by wet granulation, dry granulation or direct compression techniques. Capsules can be made by mixing all ingredients, optionally granulating the mix using wet or dry granulation or spheronizing and pelletizing and filling in empty hard gelatin or HPMC capsule shells. The capsules may also be in the form of soft gelatin capsules wherein the all the ingredients are dispersed or dissolved in a suitable medium. Liquid solutions can be prepared by dissolving the drug and other excipients in a suitable medium.

In a preferred embodiment, the stabilized tablet composition comprising pramipexole can be prepared using dry granulation, wet granulation or direct compression. Dry granulation may be carried out, for example, by using a roller compactor or alternatively, for example, by the process of slugging. Wet granulation may be carried out, using aqueous and/or non aqueous solvents. Examples of solvents used as granulating fluids include but are not limited to methylene chloride, isopropyl alcohol, acetone, methanol, ethanol, water or mixtures thereof.

In a still preferred embodiment the stabilized tablet composition comprising pramipexole as disclosed herein can be prepared by a process comprising dissolving pramipexole dihydrochloride in suitable solvent such as water along with polyvinyl pyrrolidone. A blend of β-cyclodextrin and other excipients is then granulated with the above solution. The granules are dried and sifted through suitable mesh. Then granules are lubricated using colloidal silicon dioxide and magnesium sterate. This lubricated blend is then compressed using suitable tablet equipment. The composition can be packaged using different packaging materials well known to persons skilled in the art e.g. blisters, HDPE containers and the like.

In another embodiment the stabilized tablet composition of pramipexole disclosed as herein can be prepared by a process comprising the preparation of inclusion complex of pramipexole dihydrochloride with β-cyclodextrin in molar ratio 1: (0.25-4), preferably 1:1, preferably using kneading method well known to persons skilled in the art. The inclusion complex prepared is then admixed with suitable conventional excipients. The granulation is then carried out using either dry granulation process or wet granulation process which may be as aqueous or non-aqueous. These granules are further dried, sifted and lubricated. This lubricated blend is then compressed using suitable tablet equipment. The composition can be packaged using different packaging material well known to persons skilled in the art e.g. blisters, HDPE containers and the like.

In another preferred embodiment the stabilized pharmaceutical compositions can be packaged in a suitable packaging system which can also comprise means for reducing the oxygen content of the packaging system containing the stabilized compositions. Examples of such means may include but are not limited to oxygen absorbers, inert gases such as nitrogen, argon and the like or combinations thereof. Oxygen absorbers reduce the oxygen concentration in a sealed container creating a very low-oxygen environment. Examples of oxygen absorbers which are commercially available include but are not limited to O-busters® and the like. These means can be introduced into the packaging system containing the stabilized compositions using techniques and equipments well known to those skilled in the art.

Thus in a preferred aspect there is provided a stabilized tablet formulation comprising pramipexole or pharmaceutically acceptable salts thereof and β-cyclodextrin that further comprises one or more pharmaceutically acceptable excipients wherein the stabilized tablet composition is further packed in a suitable container which also comprises means of reducing the oxygen content in the container. Examples of such mean include but are not limited to oxygen absorbers, inert gases such as nitrogen, argon and the like or combinations thereof.

In a still preferred embodiment a stabilized tablet composition, wherein said composition comprises, based on weight: a) pramipexole dihydrochloride 0.15%, b) β-cyclodextrin 27.0%, c) maize starch 35%, d) polyvinyl pyrrolidone 2.0%, d) microcrystalline cellulose 33.30%, e) colloidal silicon dioxide 1.10% and f) magnesium stearate 1.45% wherein tablet composition is packaged in a suitable packaging system which comprises means for reducing the oxygen content such as oxygen absorbers or inert gases such as nitrogen, argon or combinations thereof in the packaging system containing the stabilized composition.

In a still preferred embodiment a stabilized tablet composition comprising pramipexole and β-cyclodextrin may be packaged in inner black lined HDPE containers which optionally comprise of means for reducing the oxygen content such as oxygen absorbers or inert gases such as nitrogen, argon or combinations thereof.

Stability studies of the tablet compositions of this invention along with suitable tablet compositions as reference were conducted by storage from periods up to twenty four weeks at various accelerated stability conditions such as 40° C. with 75% relative humidity; and/or at 50° C. and using different packaging.

While the present invention has been described in terms of its specific embodiments, certain modifications and equivalents will be apparent to those skilled in the art and are included within the scope of the present invention. Examples are provided to illustrate particular aspects of the disclosure and do not limit the scope of the present invention as defined by the claims.

### EXAMPLE 1

Pramipexole dihydrochloride tablets were prepared having compositions shown in Table 1. Pramipexole dihydrochloride was dissolved in purified water along with polyvinyl pyrrolidone. Mannitol and/or β-cyclodextrin and/or maize starch were mixed and granulated with above solution. The granules were dried at 50-60° C. and sifted through suitable mesh. The granules were lubricated using colloidal silicon dioxide and magnesium stearate. This lubricated blend was compressed using suitable tablet press.

TABLE 1
Composition of pramipexole tablets.

| | Percent (%) quantity per tablet | | | | |
|---|---|---|---|---|---|
| | Formulation Name | | | | |
| Ingredients | O | A | B | C | D |
| Pramipexole | 0.15 | 0.15 | 0.15 | 0.15 | 0.15 |
| Dihydrochloride | | | | | |
| Mannitol | 59.85 | 58.85 | 54.85 | — | — |
| β-cyclodextrin | 0 | 1 | 5 | 59.85 | 94.85 |
| Maize starch | 35 | 35 | 35 | 35 | — |
| Polyvinyl pyrrolidone | 2 | 2 | 2 | 2 | 2 |
| (Povidone) | | | | | |
| Colloidal silicon | 1.5 | 1.5 | 1.5 | 1.5 | 1.5 |
| dioxide | | | | | |
| Magnesium stearate | 1.5 | 1.5 | 1.5 | 1.5 | 1.5 |

Accelerated stability studies were conducted for compositions of Example 1 at 40° C. with 75% relative humidity; and at 50° C. Results are depicted below in Table 2.

TABLE 2
Accelerated stability data

| | % Potency | | | | | % Potency | | | | |
|---|---|---|---|---|---|---|---|---|---|---|
| | 40° C./75 RH | | | | | 50° C. | | | | |
| Period | Formulation Name | | | | | | | | | |
| (No. of Weeks) | O | A | B | C | D | O | A | B | C | D |
| % Concentration of | 0 | 1 | 5 | 60 | 94.85 | 0 | 1 | 5 | 60 | 94.85 |
| Cyclodextrin in | | | | | | | | | | |
| formulation | | | | | | | | | | |
| 0 | 101.0 | 101.1 | 101.9 | 102.7 | 103.5 | 101.1 | 101.0 | 101.0 | 101.9 | 103.1 |
| 2 | 99.1 | 100.8 | 101.5 | 102.6 | 103.5 | 94.8 | 100.4 | 100.6 | 101.5 | 103.1 |
| 4 | 98.2 | 99.7 | 100.1 | 101.5 | 103.2 | 92.9 | 98.6 | 99.0 | 99.6 | 102.1 |
| 8 | 96.1 | 99.0 | 100.0 | 100.8 | 103.0 | — | — | — | — | — |
| 12 | 93.5 | 98.6 | 99.2 | 99.7 | 103.0 | — | — | — | — | — |
| 24 | 90.1 | 98.3 | 98.7 | 99.1 | 102.3 | — | — | — | — | — |

From the results tabulated in Table 2 it can be concluded that increase in percentage of cyclodextrin in the formulation increases the stability of the formulation. All the formulations with cyclodextrin showed enhanced stability as seen in terms of percentage potency retained as compared to the formulation without cyclodextrin (i.e. 0) ranging from 98.3% to 102.3% at 40° C./75 RH after 24 weeks and from 98.6% to 102.1% at 50° C. after 4 weeks.

### EXAMPLE 2

Pramipexole dihydrochloride tablets were prepared having composition shown in Table 3. The inclusion complex of pramipexole dihydrochloride with β-cyclodextrin at molar ratio 1:1 was prepared using kneading method. The inclusion complex prepared was then admixed with suitable conventional excipients. The granulation was carried out using non-aqueous ‘wet granulation’ process. These granules were further dried, sifted through suitable mesh and lubricated. This lubricated blend was compressed using suitable tablet press.

TABLE 3
Composition of pramipexole tablets

Percent (%) quantity per tablet

| Ingredients | E | F |
|---|---|---|
| Pramipexole dihydrochloride: | 0.75 | 2.50 |
| β-cyclodextrin inclusion | | |
| complex [molar ratio 1:1] | | |
| Mannitol | 59.25 | 57.50 |
| Maize starch | 35 | 35 |
| Polyvinyl pyrrolidone | 2 | 2 |
| (Povidone) | | |
| Colloidal silicon dioxide | 1.5 | 1.5 |
| Magnesium stearate | 1.5 | 1.5 |

Accelerated stability studies were performed for the compositions of Example 2 at 40° C. with 75% relative humidity; and at 50° C. Results are presented in Table 4.

TABLE 4
Accelerated stability data

| | % Potency | | | % Potency | | |
|---|---|---|---|---|---|---|
| | 40° C./75 RH | | | 50° C. | | |
| Period | Formulation Name | | | | | |
| (No. of Weeks) | O | E | F | O | E | F |
| Pramipexole dihydrochloride: | 0 | 0.75 | 2.50 | 0 | 0.75 | 2.50 |
| β-cyclodextrin Inclusion | | | | | | |
| Complex [molar ratio 1:1] % | | | | | | |
| concentration in formulation. | | | | | | |
| 0 | 101.0 | 103.28 | 103.30 | 101.1 | 104.31 | 104.27 |
| 2 | 99.1 | 103.20 | 103.29 | 94.8 | 104.30 | 104.26 |
| 4 | 98.2 | 103.16 | 103.27 | 92.9 | 104.28 | 104.26 |
| 8 | 96.1 | 102.96 | 103.27 | — | — | — |
| 12 | 93.5 | 102.00 | 103.26 | — | — | — |

From the results tabulated in Table 4 it can be concluded that increase in percentage of inclusion complex in the formulation increases stability of the formulations as compared to the formulation without cyclodextrin inclusion complex (i.e. O).

The compositions of Example 2 were also subjected to stability studies at 40° C. with 75% relative humidity; and at 50° C. with oxygen absorbers and nitrogen inside the containers containing the compositions. Results are presented in Table 5 and Table 6 for oxygen absorbers and nitrogen respectively.

TABLE 5
Accelerated stability data with packaging variables.

| % Degradation | % Degradation |
|---|---|
| 40° C./75 RH | 50° C. |
| Packaging Variable | |
| With oxygen | With oxygen |
| absorbers | absorbers |

| | |
|---|---|
| Period (No. | Formulation Name |

| of Weeks) | O | F | O | F |
|---|---|---|---|---|
| Pramipexole | 0 | 2.50 | 0 | 2.50 |
| dihydrochloride: | | | | |
| β-cyclodextrin | | | | |
| inclusion complex | | | | |
| [mole ratio 1:1] | | | | |
| % concentration in | | | | |
| formulation. | | | | |
| 2 | 1.45 | 1.27 | 4.73 | 0.58 |
| 4 | 2.83 | 2.05 | 6.12 | 0.97 |
| 8 | 4.67 | 2.88 | — | — |
| 12 | 5.97 | 3.16 | — | — |

TABLE 6
Accelerated stability data with packaging variables.

| % Degradation | % Degradation |
|---|---|
| 40° C./75 RH | 50° C. |
| Packaging Variable | |
| With Nitrogen | With Nitrogen |
| flushing | flushing |

| | |
|---|---|
| Period (No. | Formulation Name |

| of Weeks) | O | F | O | F |
|---|---|---|---|---|
| Pramipexole | 0 | 2.50 | 0 | 2.50 |
| dihydrochloride: | | | | |
| β-cyclodextrin | | | | |
| inclusion iomplex | | | | |
| [mole ratio 1:1] % | | | | |
| concentration in | | | | |
| formulation. | | | | |
| 2 | 1.48 | 1.23 | 4.97 | 0.75 |
| 4 | 2.96 | 1.87 | 6.27 | 1.18 |
| 8 | 4.73 | 2.68 | — | — |
| 12 | 6.23 | 3.02 | — | — |

The results of Table 5 and Table 6 suggest that packaging of stabilized compositions with oxygen absorbers and nitrogen further enhances stability of the compositions.

### EXAMPLE 3

Pramipexole dihydrochloride tablets were prepared having composition shown in Table 7. Pramipexole dihydrochloride was dissolved in purified water along with polyvinyl pyrrolidone. β-cyclodextrin and/or mannitol and/or maize starch were mixed and granulated with the above solution. The granules were dried at 50-60° C. and sifted through suitable mesh. The granules were lubricated with colloidal silicon dioxide and magnesium stearate. This lubricated blend was compressed using suitable tablet press.

TABLE 7
Composition of pramipexole tablets.

| | Percent (%) quantity per tablet | | | | |
|---|---|---|---|---|---|
| | Formulation Name | | | | |
| Ingredients | O1 | A1 | B1 | C1 | D1 |
| Pramipexole | 0.15 | 0.15 | 0.15 | 0.15 | 0.15 |
| Dihydrochloride | | | | | |
| Mannitol | 60.0 | — | — | — | — |
| β-cyclodextrin | — | 60.0 | 27.0 | 60.0 | 94.85 |
| Maize starch | 35 | — | 35 | 35.45 | — |
| Polyvinyl pyrrolidone | 2 | 2.0 | 2.0 | 2.0 | 2 |
| (Povidone) | | | | | |
| Microcrystalline | — | 35.45 | 33.30 | — | — |
| cellulose | | | | | |
| Colloidal silicon | 1.5 | 1.10 | 1.10 | 1.10 | 1.5 |
| dioxide | | | | | |
| Magnesium stearate | 1.5 | 1.45 | 1.45 | 1.45 | 1.5 |

The compositions of Table 7 were subjected to accelerated stability studies at 40° C. with 75% relative humidity; and at 50° C. Results are tabulated in Table 8.

TABLE 8
Accelerated stability data for composition O1 to D1

| | % Degradation | | | | | % Degradation | | | | |
|---|---|---|---|---|---|---|---|---|---|---|
| | 40° C./75 RH | | | | | 50° C. | | | | |
| Period | Formulation Name | | | | | | | | | |
| (No. of Weeks) | O1 | A1 | B1 | C1 | D1 | O1 | A1 | B1 | C1 | D1 |
| % Concentration of | 0 | 60.0 | 27.00 | 60.0 | 94.85 | 0 | 60.0 | 27.00 | 60.0 | 94.85 |
| Cyclodextrin in | | | | | | | | | | |
| formulation | | | | | | | | | | |
| 2 | 1.88 | 1.53 | 0.07 | 0.23 | 0.00 | 6.14 | 2.82 | 0.10 | 1.07 | 0.39 |
| 4 | 2.77 | 2.91 | 0.10 | 0.39 | 0.29 | 8.02 | 5.35 | 0.40 | 1.17 | 1.35 |
| 8 | 4.85 | 3.85 | 1.71 | 1.17 | 0.48 | — | — | — | — | — |
| 12 | 7.42 | 4.79 | 2.61 | 2.54 | 0.48 | — | — | — | — | — |
| 24 | 10.79 | 4.32 | 3.50 | 3.22 | 1.16 | — | — | — | — | — |

From the results presented in the above table it can be concluded that increase in percentage of cyclodextrin in the formulation increases stability of the formulation. All the formulations with cyclodextrin showed enhanced stability as compared to the formulation without cyclodextrin (i.e. O1) with percentage degradation of pramipexole being from 4.32% to 1.16% at 40° C./75 RH after 24 weeks and from 5.35% to 1.35% at 50° C. after 4 weeks.

The compositions of Example 3 were also subjected to accelerated stability studies at 40° C./75 RH and 50° C. with oxygen absorbers and nitrogen in the primary containers containing the compositions. The results of these studies are presented in Table 9 and Table 10 for oxygen absorbers and nitrogen respectively.

TABLE 9
Accelerated stability data of with packaging variables.

| % Degradation | % Degradation |
|---|---|
| 40° C./75 RH | 50° C. |
| Packaging Variable | |
| With oxygen | With oxygen |
| absorbers | absorbers |

| | |
|---|---|
| Period (No. | Formulation Name |

| of Weeks) | O1 | B1 | O1 | B1 |
|---|---|---|---|---|
| % Concentration of | 0 | 27.0 | 0 | 27.0 |
| Cyclodextrin in | | | | |
| formulation | | | | |
| 2 | 0.97 | 0.13 | 4.75 | Nil |
| 4 | 1.87 | 0.95 | 6.12 | 0.2 |
| 8 | 4.13 | 1.36 | — | — |
| 12 | 5.93 | 1.57 | — | — |
| 24 | 7.12 | 1.98 | — | — |

TABLE 10
Accelerated stability data with packaging variables.

| % Degradation | % Degradation |
|---|---|
| 40° C./75 RH | 50° C. |
| Packaging Variable | |
| With Nitrogen | With Nitrogen |
| flushing | flushing |

| | |
|---|---|
| Period (No. | Formulation Name |

| of Weeks) | O | B | O | B |
|---|---|---|---|---|
| % Concentration of | 0 | 27.0 | 0 | 27.0 |
| Cyclodextrin in | | | | |
| formulation | | | | |
| 2 week | 1.13 | 0.17 | 4.28 | Nil |
| 4 week | 2.27 | 1.05 | 5.95 | 0.2 |
| 8 week | 5.07 | 1.55 | — | — |
| 12 week | 7.17 | 1.93 | — | — |
| 24 week | 8.90 | 2.52 | — | — |

From the results presented above it can be concluded that the packaging in presence of oxygen absorbers and nitrogen enhances stability of the compositions.

Thus in conclusion pramipexole dihydrochloride tablets with β-cyclodextrin as co-excipient and inclusion complex showed lesser degradation of pramipexole dihydrochloride at accelerated stability conditions in comparison to the formulations without β-cyclodextrin. Also pramipexole dihydrochloride tablets with β-cyclodextrin as co-excipient and inclusion complex with packaging using nitrogen purging and oxygen absorbers showed further stability enhancement in comparison to formulations without β-cyclodextrin.

1. A stabilized pharmaceutical composition comprising pramipexole or a pharmaceutically acceptable salt thereof and one or more dextrins.

2. A stabilized pharmaceutical composition according to claim 1 wherein, pramipexole is pramipexole dihydrochloride.

3. A stabilized pharmaceutical composition according to claim 1 wherein, the dextrin present in a level between 0.01% to 95% w/w of the composition.

4. A stabilized pharmaceutical composition according to claim 1 wherein, the dextrin is cyclodextrin.

5. A stabilized pharmaceutical composition according to claim 4 wherein, cyclodextrin is present as co-excipient.

6. A stabilized pharmaceutical composition according to claim 5 wherein, cyclodextrin is present in a level between 1% to 60% w/w of composition.

7. A stabilized pharmaceutical composition according to claim 5 wherein, cyclodextrin is present in a level between 20% to 40% w/w of composition.

8. A stabilized pharmaceutical composition according to claim 4 wherein, cyclodextrin is present as inclusion complex.

9. A stabilized pharmaceutical composition according to claim 8 wherein the molar ratio of pramipexole to cyclodextrin in the inclusion complex is between 1:0.25 to 1:4.

10. A stabilized pharmaceutical composition according to claim 8 wherein, the molar ratio of pramipexole to cyclodextrin in the inclusion complex is 1:1.

11. A stabilized pharmaceutical composition according to claim 8 wherein, the inclusion complex is present in a level between 0.01% to 95% w/w of composition.

12. The stabilized pharmaceutical composition according to claim 4 wherein cyclodextrin is selected from α-cyclodextrin, β-cyclodextrin, γ-cyclodextrin, alkyl or hydroxyalkyl derivatives thereof, such as (2,6-di-o-methyl)-β-cyclodextrin, randomly methylated β-cyclodextrin and hydroxypropyl β-cyclodextrin and sulpho-butyl-ether β-cyclodextrin.

13. The stabilized pharmaceutical composition according to claim 4 wherein cyclodextrin is β-cyclodextrin.

14. The stabilized pharmaceutical composition according to claim 1 wherein the composition is in the form of a tablet, capsule, multiparticulate system, granule, powder.

15. The stabilized pharmaceutical composition according to claim 1 wherein the composition is in the form of a tablet.

16. The stabilized tablet composition according to claim 15 comprising;
a) pramipexole dihydrochloride
b) β-cyclodextrin and;
c) one or more pharmaceutically acceptable excipients

17. A process for preparing a stabilized tablet composition according to claim 15, wherein, the process comprising;
a) dissolving pramipexole dihydrochloride along with polyvinyl pyrrolidone in suitable solvent;
b) granulating blend of cyclodextrin and other excipients with above solution as granulating fluid;
c) drying of above formed granules;
d) lubricating granules with glidants and anti-adherents;
e) compressing granules using suitable tablet equipment.

18. A process of preparing a stabilized tablet composition according to claim 15, wherein, the process comprising;
a) preparing pramipexole dihydrochloride-β-cyclodextrin inclusion complex;
b) admixing prepared inclusion complex with other excipients;
c) granulating using either dry granulation process or wet granulation process or direct compression;
d) drying, sifting and lubricating, formed granules;
e) compressing granules using suitable tablet equipment to form tablet.

19. A method of treating diseases in a mammal in need thereof wherein pramipexole or pharmaceutically acceptable salts thereof are effective by administering to the mammal a stabilized pharmaceutical composition according to claim 1.

20. A method of packaging a stabilized pharmaceutical composition according to claim 1 comprising including oxygen absorbers in the packaging system comprising the composition.

21. A method of packaging stabilized pharmaceutical composition according to claim 1 comprising including an inert gas in the packaging system comprising the composition.

22. The packaging system of claim 21 wherein, the inert gas is selected from the group consisting of nitrogen and argon.

23. A stabilized tablet composition, comprising,
a) pramipexole dihydrochloride
b) β-cyclodextrin
c) maize starch
d) polyvinyl pyrrolidone
e) microcrystalline cellulose
f) colloidal silicon dioxide and
g) magnesium stearate;
wherein, the tablet composition is further packaged in a packaging system comprising of oxygen absorbers or inert gases or combination thereof.

24. A stabilized tablet composition according to claim 23 wherein, the inert gas is nitrogen.
